(19) United States
(12) Patent Application Publication
(10) Pub. No.: US 2010/0152601 A1
(43) Pub. Date: Jun. 17, 2010

(54) ASPIRATION STATE MONITORING DEVICE, ASPIRATION STATE MONITORING SYSTEM, BASE DEVICE, CONTROL SYSTEM, AND CONTROL SYSTEM USE METHOD

(75) Inventors: Junichi Akiyama, Nagoya-shi (JP); Katsuhiko Horii, Nagoya-shi (JP); Hiroyuki Nagatani, Nagoya-shi (JP); Atsushi Wakita, Nagoya-shi (JP)

Correspondence Address: SUGHRUE MION, PLLC, 2100 PENNSYLVANIA AVENUE, N.W., SUITE 800, WASHINGTON, DC, 20037, US

(73) Assignee: NGK SPARK PLUG CO., LTD., Nagoya-shi, Aichi (JP)

(21) Appl. No.: 11/993,473

(22) PCT Filed: Jun. 20, 2006
(86) PCT No.: PCT/JP2006/312345

§ 371 Date: Dec. 21, 2007

(30) Foreign Application Priority Data
Jun. 24, 2005 (JP) 2005-185412
Jun. 24, 2005 (JP) 2005-185413

(51) Int. Cl. A61B 5/08

(52) U.S. Cl. 600/538

(57) ABSTRACT

Disclosed is an aspiration state monitoring device including a rechargeable battery, a memory that stores aspiration data indicating aspiration state of a living body, and a monitoring side attachment unit detachably attached to a base device. The monitoring side attachment unit has a monitoring side charge conducting section that receives electric power from the base device and charges the rechargeable battery, and a monitoring side communication conducting section which transmits the aspiration data stored in the memory to the base device.

### TECHNICAL FIELD

The present invention relates to an aspiration state monitoring device, an aspiration state monitoring system, a base device, a control system, and a control system use method, capable of examining aspiration state such as normal or abnormal state of aspiration and snoring during sleep, for example, by using a piezoelectric film or others.

### BACKGROUND ART

Currently, a technology of attaching a thermistor to a person around his or her mouth to examine aspiration state is known (see Patent Reference 1).

Also, a technology of detecting body actions by a piezoelectric element to examine aspiration state based on detection result is known (see Patent Reference 2).

In recent years, a technology of attaching a PVDF (polyvinylidene fluoride) film to a person's mouth and detecting aspiration based on a detection signal from the film has also been disclosed (see Patent Reference 3).

Patent Reference 1: Japanese Patent No. 2,794,196 (page 2, FIG. 2)

Patent Reference 2: Japanese Patent No. 2,803,374 (page 1, FIG. 2)

Patent Reference 3: U.S. Pat. No. 5,311,875 (page 1, FIG. 1)

### DISCLOSURE OF THE INVENTION

### Problems to be Solved by the Invention

According to the technologies disclosed in the above references, a sensor for detecting aspiration state is connected with a measuring device during use. However, this measuring device is large-sized and is difficult to be used for screening.

That is, for easy detection of abnormal aspiration state or the like of a number of examinees to each of which the measuring device is attached, improvements such as device size reduction and enhanced operability are needed. However, there is a problem that measures taken for these improvements have not been sufficient.

Particularly in case of measurement by a device made compact and attached to each person, no consideration has been given to a system which collects data from each device and executes screening under the present situation.

The invention has been developed to solve the problems discussed above. It is an object of the invention to provide an aspiration state monitoring device, an aspiration state monitoring system, a base device, a control system, and a control system use method, which can operate easily, execute screening preferably, and achieve device size reduction and cost reduction.

### Means to Solve the Problem

(1) The invention of claim 1 pertains to an aspiration state monitoring device including a rechargeable battery, a memory (such as flash memory) that stores aspiration data indicating aspiration state (such as presence or absence of aspiration and snoring) of a living body, and a monitoring side attachment unit detachably attached to a base device. According to the invention, the monitoring side attachment unit has a monitoring side charge conducting section (such as a terminal of a connector) that receives electric power from the base device and charges the rechargeable battery, and a monitoring side communication conducting section (such as another terminal of the connector) which transmits the aspiration data stored in the memory to the base device.

Thus, according to this invention, charging and aspiration data transmission and reception can be easily achieved by attaching the aspiration state monitoring device to the base device.

That is, at the time of screening for many examinees by using a number of the aspiration state monitoring devices, charging and aspiration data transmission and reception can be easily achieved by attaching the aspiration state monitoring device after measurement to the base device. Accordingly, a significant advantage that screening can be performed by extremely easy operation with highly enhanced screening efficiency can be provided.

(2) According to the invention of claim 2, the monitoring side attachment unit is a connector on the aspiration state monitoring device side (such as a device side lower connector) connected with a connector on the base device side (such as a base side connector).

This invention exemplifies the monitoring side attachment unit. In this case, charging and data transmission and reception can be easily executed by connecting both the connectors.

(3) According to the invention of claim 3, one connector of the aspiration state monitoring device has a terminal of the monitoring side charge conducting section (such as a terminal of the connector) and a terminal of the monitoring side communication conducting section (such as another terminal of the connector).

This invention exemplifies a structure of the terminals contained in the connector. In this case, charging and data transmission and reception can be easily executed only by connecting the pair of the connectors.

(4) According to the invention of claim 4, a barcode indicating an ID of the aspiration state monitoring device is provided on a surface of the aspiration state monitoring device.

Thus, it is possible to determine which of the aspiration state monitoring device among multiple aspiration state monitoring devices is to be processed by input of the ID data to the base device side.

(5) According to the invention of claim 5, the memory of the aspiration state monitoring device (such as non-volatile memory) stores ID data indicating an ID of the aspiration state monitoring device.

Thus, it is possible to determine which of the aspiration state monitoring device among the multiple aspiration state monitoring devices is to be processed by input of the ID data to the base device side.

(6) The invention of claim 6 pertains to a base device which includes a base side attachment unit (such as base side connector) to which a plurality of aspiration state monitoring devices, each of which includes a rechargeable battery and a memory storing aspiration data indicating aspiration state of a living body, are detachably attached. The base side attachment unit according to this invention has a base side charge conducting section (such as a terminal of the connector) that charges the rechargeable battery of each of the aspiration state monitoring devices, and a base side communication conducting section (such as another terminal of the connector) which collects the aspiration data stored in the memory of each of the aspiration state monitoring devices.

According to this invention, therefore, charging and aspiration data transmission and reception can be easily executed by attaching the aspiration state monitoring device to the base device, or by attaching the plural aspiration state monitoring devices to the corresponding plural base side attachment units of the base device, for example.

Thus, in case of screening for many examinees by using a number of the aspiration state monitoring devices, charging and aspiration data transmission and reception can be executed by attaching the aspiration state monitoring devices after measurement to the base device. Accordingly, a significant advantage that screening can be performed by extremely easy operation with highly enhanced screening efficiency can be provided.

(7) According to the invention of claim 7, the base side attachment unit is a connector on the base device side connected with a connector on the aspiration state monitoring device side.

This invention exemplifies the base side attachment unit. Thus, charging and data transmission and reception can be easily executed by connecting both the connectors.

(8) According to the invention of claim 8, one connector of the base device has a terminal of the base side charge conducting section and a terminal of the base side communication conducting section.

This invention exemplifies structures of the terminals included in the connector. In this case, charging and data transmission and reception can be easily executed only by connecting the pair of the connectors.

(9) According to the invention of claim 9, the base device is divided into a plurality of blocks, and each of the blocks has a plurality of base side attachment units to which the respective aspiration state monitoring devices are detachably attached.

Thus, in case of failure in the base side attachment unit, a circuit or the like within a certain block, for example, only the corresponding block needs to be removed for repair or the like. Accordingly, maintenance is simplified.

(10) According to the invention of claim 10, a controller which controls the charging and the data collection is provided on the base device.

According to this invention, the controller is equipped integrally with or separately from the base device. In case of the separate controller, device part to which the aspiration state monitoring device is attached and the controller (such as personal computer) are connected by a cable, for example. The controller provided as a separate component is included in the concept of the base device of this invention.

Thus, control over the charging and the data collection can be achieved by operating the base device.

(11) According to the invention of claim 11, when ID data in a barcode provided on the aspiration state monitoring device to indicate an ID of the aspiration state monitoring device is read by the controller and when ID data stored in the memory of the aspiration state monitoring device to indicate the ID of the aspiration device monitoring device is read by the controller, the ID data in the barcode and the ID data in the memory are compared with each other.

According to this invention, it is possible to check whether the aspiration state monitoring device whose barcode has been read coincides with the aspiration state monitoring device which is going to start charging and data transmission and reception. Thus, the advantage that operation error, such as mistaking another person's data for a correct data, is prevented can be provided.

(12) According to the invention of claim 12, when ID data in a barcode provided on the aspiration state monitoring device to indicate an ID of the aspiration state monitoring device is read by the controller, a file name of a file for the aspiration data is automatically created based on the ID data and measurement date of the aspiration data.

Accordingly, the file name of the file for storing the aspiration data, for example a file name containing the ID and date, is automatically created based on the ID data and date. Thus, an operation error, such as mistaking another person's data for a correct data, can be prevented.

(13) According to the invention of claim 13, when ID data stored in the memory of the aspiration state monitoring device to indicate an ID of the aspiration state monitoring device is read by the controller, a file name of a file for the aspiration data is automatically created based on the ID data and the measurement date of the aspiration data.

Accordingly, the file name of the file for storing the aspiration data, for example a file name containing the ID and date, is automatically created based on the ID data and date. Thus, an operation error, such as mistaking another person's data for a correct data, can be prevented.

(14) The invention of claim 14 is a control system comprising the aspiration state monitoring device according to any one of claims 1 through 5, and the base device according to any one of claims 6 though 13.

According to this invention, screening can be easily and efficiently performed by attaching the multiple aspiration state monitoring devices to the base device.

(15) The invention of claim 15 is a method of using the control system according to claim 14. According to this invention, the aspiration state monitoring device is attached to the base device. The rechargeable battery of the aspiration state monitoring device is charged by the base device via the base side charge conducting section and the monitoring side charge conducting section. The aspiration data is collected from the memory of the aspiration state monitoring device by the base device via the monitoring side communication conducting section and the base side communication conducting section.

According to this invention, it is possible to supply electric power from the base device to the rechargeable battery of the aspiration state monitoring device and charge the rechargeable battery by attaching the aspiration state monitoring device to the base device. In addition, it is possible to transmit aspiration data from the memory of the aspiration state monitoring device to the base device and collect aspiration data.

(16) The invention of claim 16 is a method of using the control system according to claim 14. According to this invention, the aspiration state monitoring device is attached to the base device, and at least either initialization or time adjustment of the aspiration state monitoring device is executed by the base device via the base side charge conducting section and the monitoring side charge conducting section.

According to this invention, initialization or time adjustment (such as date setting) of the aspiration state monitoring device is executed after the aspiration state monitoring device is attached to the base device. Thus, an operation for re-using the aspiration state monitoring device can be efficiently performed.

(17) The invention of claim 17 pertains to an aspiration state monitoring device to which an aspiration sensor that outputs a signal corresponding to aspiration state of a living body (such as presence or absence of aspiration and snoring) is detachably attached to measure the aspiration state (particularly to record measurement data) based on the signal from the aspiration sensor. According to this invention, electric continuity between the battery and an internal circuit (a circuit which activates the aspiration state monitoring device) of the aspiration state monitoring device is connected or disconnected by attachment or detachment of a connector of the aspiration sensor.

According to this invention, a circuit connecting the battery and the internal circuit is closed and the aspiration state monitoring device comes to ON condition (power ON condition of the device) when the aspiration sensor is attached to the aspiration state monitoring device. On the other hand, the circuit connecting the battery and the internal circuit is opened and the aspiration state monitoring device comes to OFF condition (power OFF condition of the device) when the aspiration sensor is removed from the aspiration state monitoring device.

Thus, the aspiration state monitoring device can be automatically turned on or off by attachment or detachment of the aspiration sensor. Accordingly, the structure of the device can be simplified and made compact. Moreover, an operation can be securely performed with enhanced operability, and cost reduction can be achieved.

Particularly, in the case where the aspiration state monitoring device is charged after attached to the base device, for example, high voltage is applied to the internal circuit under the condition of connection between the battery (rechargeable battery) and the internal circuit during charging. In this case, the internal circuit or the like may be damaged.

According to this invention, however, the circuit between the battery and the internal circuit is opened when the aspiration sensor is removed after completion of aspiration state measurement. Thus, the advantage that damage of the internal circuit or the like is effectively prevented even in case of high-voltage charging can be provided. Moreover, there is less possibility that turning off of a power switch is forgotten than in case of a structure containing a separate power switch. Thus, safety increases.

(18) According to the invention of claim 18, a notifying unit that gives a notification corresponding to a use condition of the aspiration sensor when the aspiration sensor is attached to the aspiration state monitoring device is provided.

According to this invention, operation conditions are notified through flashes of a LED or by other methods according to use condition of the aspiration sensor when a measurement start switch or the like is turned on, for example, under the condition where the aspiration sensor is attached to the aspiration state monitoring device.

Thus, whether the aspiration sensor or the aspiration state monitoring device is abnormal or not or other conditions can be recognized at the time of measurement start. Accordingly, the advantage that useless measurement such as measuring all night using a damaged device is avoided can be provided.

(19) According to the invention of claim 19, contents of the notification given by the notifying unit vary according to breath inhale and exhale actions of the living body.

In this case, failure of the device can be easily recognized through flashes of the LED or the like according to breath inhale and exhale actions, for example.

(20) According to the invention of claim 20, the notification given by the notifying unit stops after elapse of a predetermined initial time from measurement start of the aspiration state monitoring device.

Since flashes or the like of the LED disturb sleep, notification continues for three minutes after start of measurement (monitoring), for example.

(21) According to the invention of claim 21, storing measurement result in a memory starts after elapse of the predetermined initial time.

This invention exemplifies storing timing in the memory.

(22) According to the invention of claim 22, magnification of an amplifier amplifying a signal from the aspiration sensor is automatically adjusted according to a condition of the signal.

In this case, amplifiers having different magnifications of amplification (signal amplification rates) are switched according to the condition of the signal (level of sensor output) for use, for example. Thus, sufficient accuracy of signal processing can be secured even when a low-cost microcomputer having low processing capability is used as a microcomputer used for control.

(23) According to the invention of claim 23, start time of the measurement is recorded in case that the measurement is measurement of the aspiration state of the living body under a condition where the aspiration sensor is attached to the aspiration state monitoring device.

In this case, the measurement start time can be accurately recognized.

(24) According to the invention of claim 24, measurement ends after elapse of a predetermined measurement time in case that the measurement is measurement of the aspiration state of the living body under a condition where the aspiration sensor is attached to the aspiration state monitoring device.

For accurate recognition of aspiration state of a living body, it is considered that measurement continuing for a predetermined period of time (such as 4 to 10 hours) is required. However, longer measurement time than this necessary time is not needed. Thus, the measurement ends after elapse of the necessary measurement time.

(25) According to the invention of claim 25, a case that accommodates the aspiration state monitoring device, and an attachment member (such as belt) that attaches the case to a living body are further provided.

In this case, the aspiration state monitoring device is accommodated in the case, and the case is attached to an arm, for example. Thus, aspiration state can be appropriately measured even during sleep or when an examinee wakes up and moves during measurement.

(26) The invention of claim 26 is an aspiration state monitoring device according to any one of claims 1 through 5, which has the characteristics of the aspiration state monitoring device according to any one of claims 17 through 25.

(27) The invention of claim 27 is an aspiration state monitoring system which has the aspiration sensor attached to the aspiration state monitoring device according to any one of claims 17 through 26.

In this case, aspiration state can be preferably measured by using the aspiration state monitoring system having the aspiration sensor attached to the aspiration state monitoring device.

### BRIEF DESCRIPTION OF THE DRAWINGS

FIG. 1 is an explanatory view illustrating an aspiration state monitoring system according to an embodiment.

FIG. 2 is an explanatory view illustrating a condition where an aspiration sensor is attached to an aspiration state monitoring device.

FIG. 3 is a perspective view of the aspiration sensor.

FIGS. 4(a) through 4(e) illustrate the aspiration state monitoring device, wherein: FIG. 4(a) is a top view; FIG. 4(b) is a front view; FIG. 4(c) is a bottom view; FIG. 4(d) is a left side view; and FIG. 4(e) is a back view.

FIG. 5 is a block diagram showing an electrical configuration of the aspiration state monitoring device.

FIG. 6(a) is an explanatory view illustrating a condition where the aspiration state monitoring device is attached to a base device, and FIG. 6(b) is an explanatory view showing pin arrangement of a base side connector.

FIG. 7 is an explanatory view schematically illustrating a sensor side connector and a device upper connector.

FIG. 8 is an explanatory view illustrating a system configuration of a control system.

FIG. 9(a) is a plan view of the base device, and FIG. 9(b) is a front view of the base device.

FIG. 10 is a block diagram showing an electrical configuration of the base device.

FIG. 11 is an explanatory view showing a case and a belt.

FIG. 12 is an explanatory view illustrating a condition where the case is attached to an arm.

FIG. 13 is an explanatory view showing a method of using an aspiration state monitoring system.

### EXPLANATION OF REFERENCES

1 . . . aspiration state monitoring system
2 . . . aspiration sensor
3 . . . aspiration state monitoring device
13 . . . sensor side connector
23 . . . piezoelectric element
29 . . . device side upper connector
31, 33, 35, 37, 101, 103 . . . LED
41 . . . measurement start switch
43 . . . base device
45 . . . device side lower connector
69 . . . rechargeable battery
71 . . . power supply circuit
72 . . . base side connector
91 . . . control system
95 . . . personal computer

### BEST MODE FOR CARRYING OUT THE INVENTION

An example of a best mode for carrying out the invention (embodiment) is hereinafter described.

### Embodiment

A device configuration and the like according to this embodiment are used for extracting (screening) aspiration abnormal state of a number of examinees.

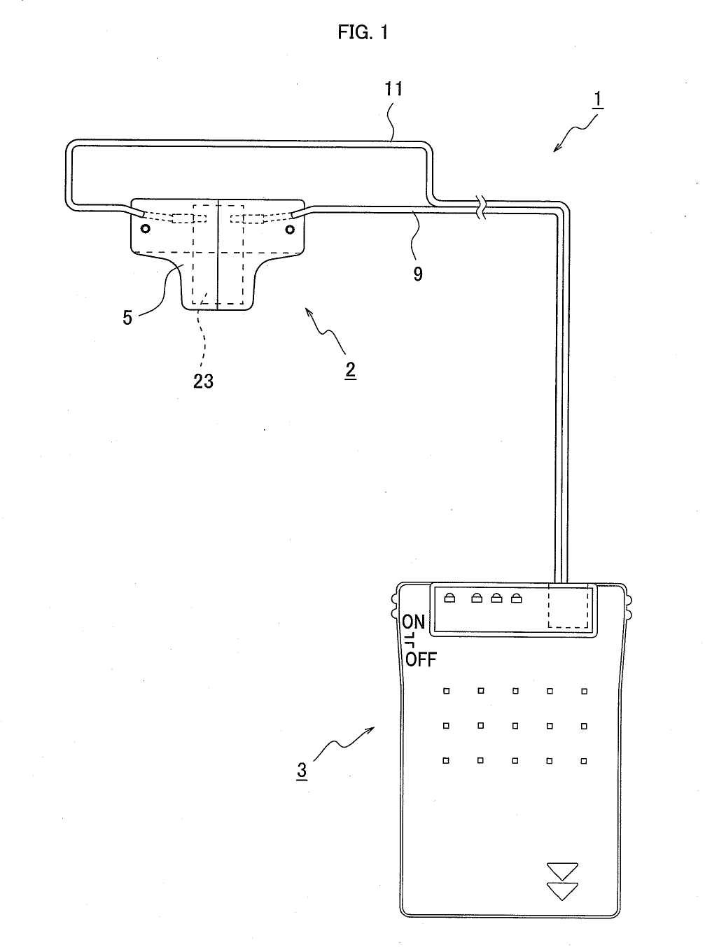

a) Initially, an aspiration state monitoring system used for monitoring aspiration state included in the device configuration according to this embodiment is discussed with reference to FIGS. 1 through 4(a)-4(e).

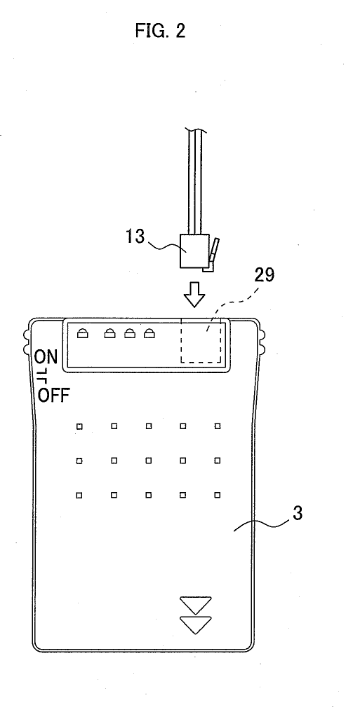

As illustrated in FIGS. 1 and 2, an aspiration state monitoring system (sleep apnea screener) 1 is a system for detecting aspiration state. More specifically, the aspiration state monitoring system 1 detects the presence or absence of aspiration during sleep. The aspiration state monitoring system 1 includes an aspiration sensor 2 attached to an examinee, and an aspiration state monitoring device 3 to which the aspiration sensor 2 is detachably attached such that the aspiration state monitoring device 3 can record a signal from the aspiration sensor 2.

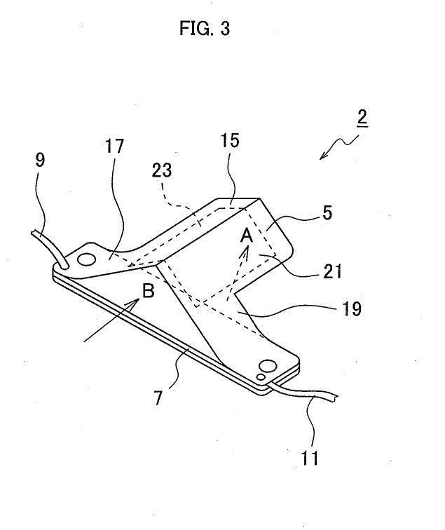

As illustrated in FIG. 3, the aspiration sensor 2 has a substantially T-shaped sensor main body 5, a connecting portion 7 connecting the left and right lower ends of the sensor main body 5, a pair of lead wires 9 and 11 extending from the sensor main body 5, and a sensor side connector 13 (see FIG. 2) attached to the tips of the lead wires 9 and 11.

The sensor main body 5 is a thin film component bended at the central portion (symmetrical axis of line symmetry for the sensor main body 5: mountain ridge of the sensor main body 5) to form a mountain shape. The sensor main body 5 has an aspiration detecting section 15 for detecting breath from a mouth and a nose, and a pair of legs 17 and 19 projecting from the left and right ends of the rear end side (lower left side in the same figure) of the aspiration detecting section 15.

The sensor main body 5 has a substantially T-shaped film base 21 made of polyester or the like, and a rectangular piezoelectric element (such as piezoelectric device) 23 attached to the inside of the slope of the mountain-shaped base (lower side in the same figure: living body side).

The aspiration detecting section 15 is bended to form a mountain shape convexed to above. The aspiration detecting section 15 is disposed in such a position as to be opposed to a flow direction of breath from the mouth (direction indicated by an arrow A). The aspiration detecting section 15 at this position contacts breath in such a manner as to block the flow of breath. The aspiration detecting section 15 is also disposed in parallel with a flow direction of breath from the nose (direction indicated by an arrow B).

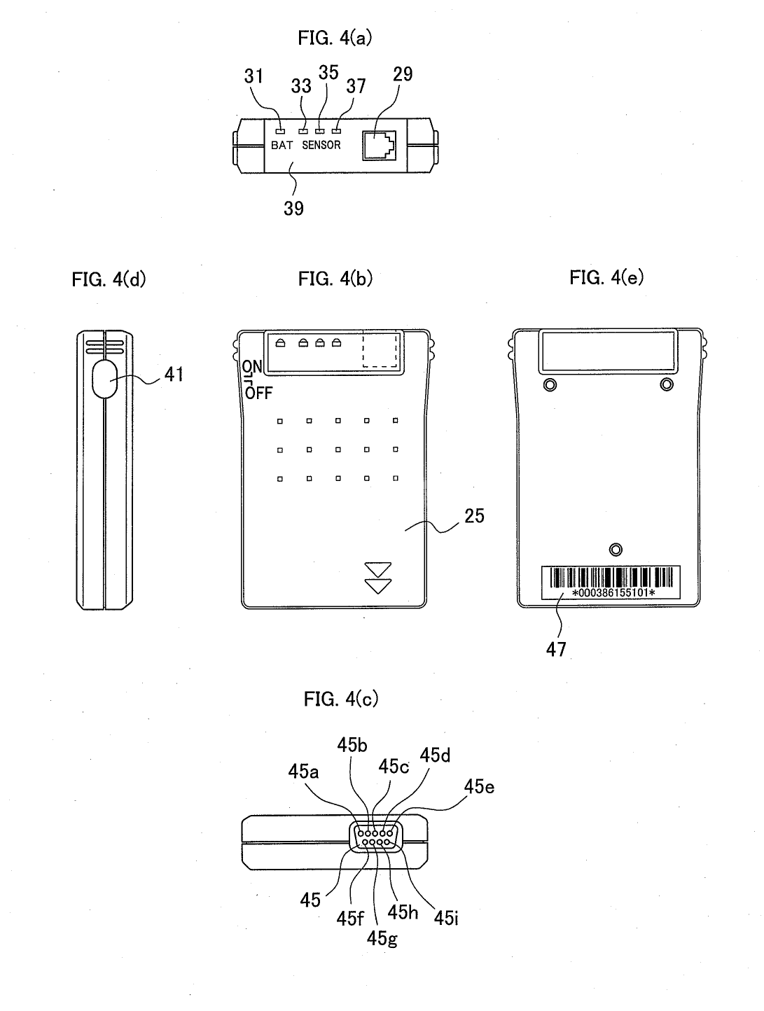

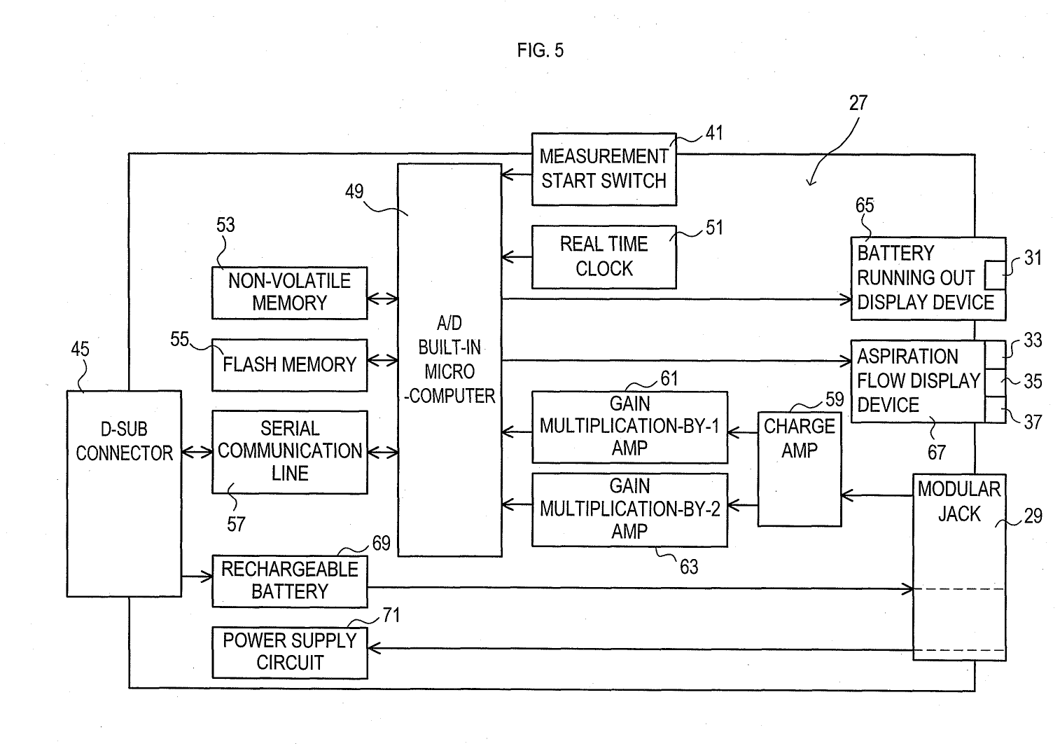

The aspiration state monitoring device 3 is a lightweight device constructed to be attachable to an arm or the like. As illustrated in FIGS. 4(a) through 4(e), the aspiration state monitoring device 3 has a housing 25 having a substantially rectangular parallelepiped shape and made of plastic material, and a monitoring side electronic controller (see FIG. 5) contained in the housing 25.

A device side upper connector (modular jack) 29 into which the sensor side connector 13 is fitted is provided on the upper surface of the aspiration state monitoring device 3. First through fourth LEDs 31 through 37 which will be described later are further provided and covered by a semi-transparent cover 39.

A measurement start switch 41 operated to turn on or off at the time of start or end of measurement (monitoring) of aspiration state is provided on the left surface of the aspiration state monitoring device 3.

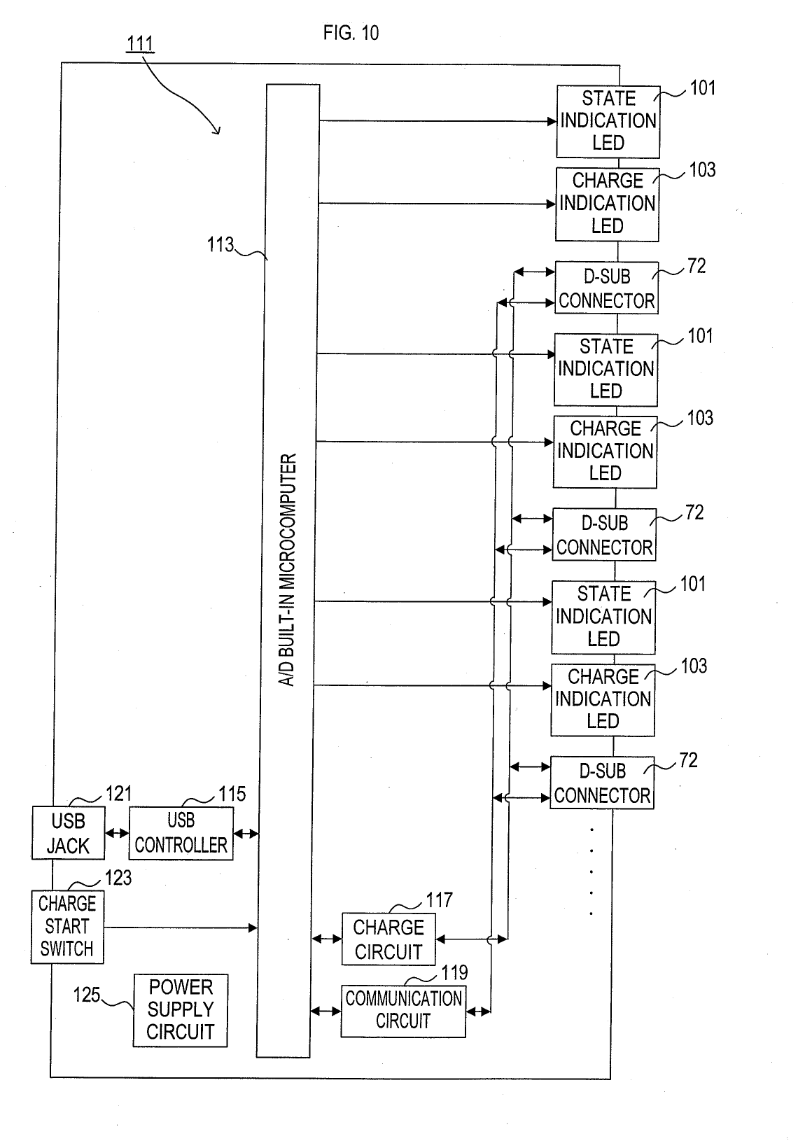

A device side lower connector (D-SUB connector: female side) 45 is equipped on the lower surface of the aspiration state monitoring device 3 to connect the aspiration state monitoring device 3 with a base device 43 (see FIG. 10).

Nine pins of terminals 45a through 45i are provided on the device side lower connector 45. Two terminals 45a and 45b of these terminals are used for charging a rechargeable battery 69 (see FIG. 5), and two other terminals 45d and 45e are used for data transmission.

A barcode sticker 47 indicating an ID number of the aspiration state monitoring device 3 is affixed to the back surface of the aspiration state monitoring device 3.

b) An electrical configuration of the aspiration state monitoring device 3 is now discussed with reference to FIGS. 5 through 7.

As illustrated in FIG. 5, the monitoring side electronic controller 27 of the aspiration state monitoring device 3 has a known A/D built-in microcomputer 49, a real time clock 51 backed up by a lithium battery, a non-volatile memory 53, a flash memory 55, a serial communication line 57, a charge amplifier (AMP) 59, a gain multiplication-by-1 AMP 61, and a gain multiplication-by-2 AMP 63.

The charge AMP 59 of the monitoring side electronic controller 27 is normally used to amplify a signal from the piezoelectric element 23. The gain multiplication-by-1 AMP 61 multiplies gain of an output from the charge AMP 59 by 1. The gain multiplication-by-2 AMP 63 multiplies gain of an output from the charge AMP 59 by 2.

The A/D built-in microcomputer 49 is connected with a first LED 31 of a battery running out display device 65 and second through fourth LEDs 33 through 37 of an aspiration flow display device 67.

The first LED 31 lights up when the voltage of the battery (rechargeable battery) 69 lowers. The second through fourth LEDs 33 through 37 flash in accordance with the aspiration state. The third LED 35 lights up when the aspiration sensor 2 is connected with the aspiration state monitoring device 3. The second LED 33 lights up when breath is inhaled. The fourth LED 37 lights up when breath is exhaled.

Particularly, in the monitoring side electronic controller 27, the device side lower connector 45 is connected with the device side upper connector 29 via the rechargeable battery 69, and the device side upper connector 29 is connected with a power supply circuit (internal circuit) 71 which supplies electric power to the monitoring side electronic controller 27.

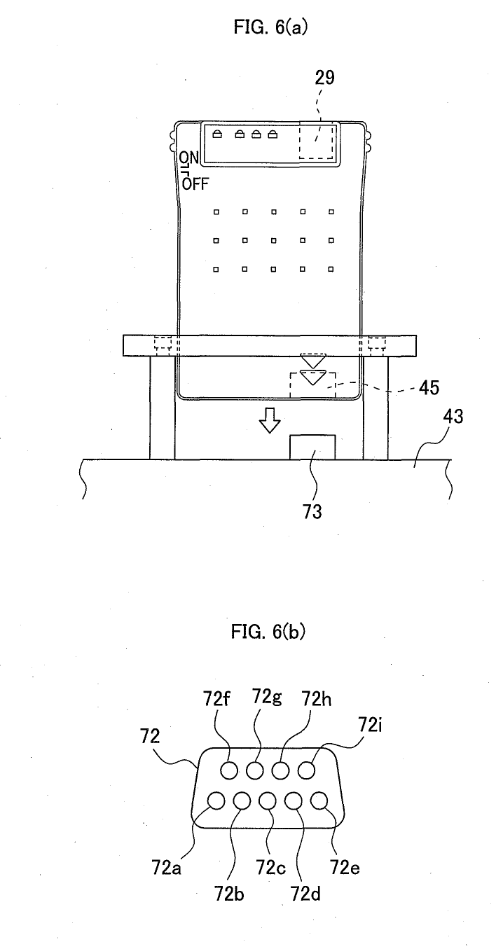

As illustrated in FIG. 6(a), the aspiration state monitoring device 3 is attached to the base device 43 such that the rechargeable battery 69 can be charged from the base side connector (D-SUB connector: male side) 72 of the base device 43 via terminals 72a and 72b exclusively used for charging on the device side lower connector (female side) 45. A circuit from the rechargeable battery 69 to the power supply circuit 71 is opened by the device side upper connector 29 in the ordinary condition (while the base device 43 is not attached).

More specifically, electric power is supplied from the rechargeable battery 69 to the power supply circuit 71 and the signal from the aspiration sensor 2 is inputted to the aspiration state monitoring device 3 by attaching the sensor side connector 13 of the aspiration sensor 2 to the device side upper connector 29.

As illustrated in FIG. 6(b), male side nine terminals 72a through 72i corresponding to the device side lower connector 45 are provided on the base side connector 72. Two terminals 72a and 72b of these terminals (connected with the two terminals 45a and 45b of the device side lower connector 45) are used for charging the rechargeable battery 69, and two other terminals 72d and 72e (connected with the two terminals 45d and 45e of the device side lower connector 45) are used for data transmission.

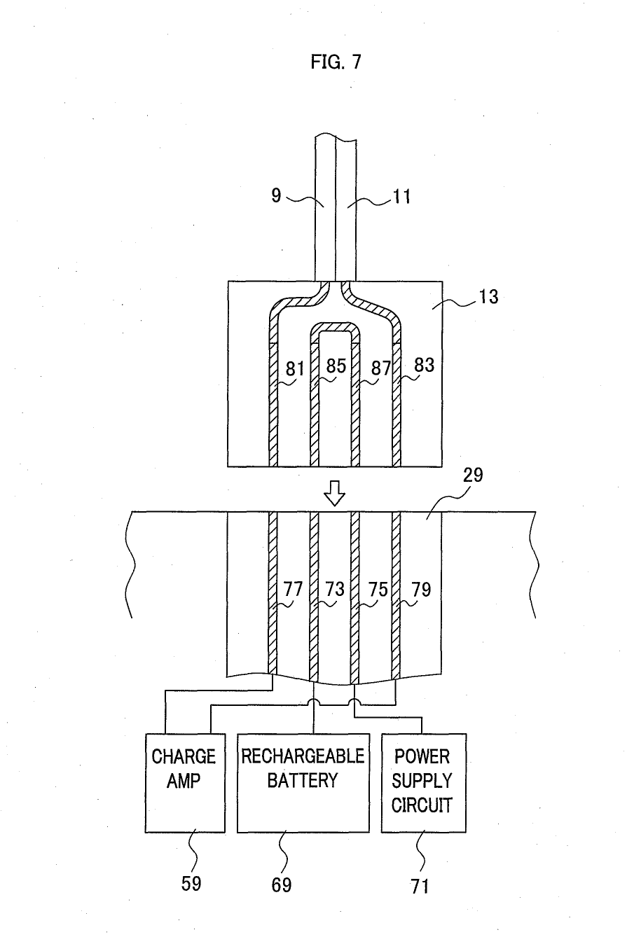

As schematically illustrated in FIG. 7, a first device side power supply terminal 73 connected with the rechargeable battery 69 and a second device side power supply terminal 75 connected with the power supply circuit 71 are disposed in parallel on the device side upper connector 29. A pair of device side signal terminals 77 and 79 connected with the charge AMP 59 are similarly disposed in parallel.

A pair of sensor side signal terminals 81 and 83 connected with the lead wires 9 and 11 in correspondence with the device side signal terminals 77 and 79 are disposed in parallel on the sensor side connector 13. First and second sensor side power supply terminals 85 and 87 are provided in correspondence with the first and second device side power supply terminals 73 and 75. These first and second sensor side power supply terminals 85 and 87 are electrically connected with each other.

Thus, when the sensor side connector 13 is fitted into the device side upper connector 29, the sensor side signal terminals 81 and 83 are brought into connection with the device side signal terminals 77 and 79. In this condition, the signal from the aspiration sensor 2 can be inputted to the aspiration state monitoring device 3.

Simultaneously, the first device side power supply terminal 73 is connected with the first sensor side power supply terminal 85, and the second device side power supply terminal 75 is connected with the second sensor side power supply terminal 87. In this condition, the circuit from the rechargeable battery 69 to the power supply circuit 71 is closed, and electric power is supplied to the monitoring side electronic controller 27.

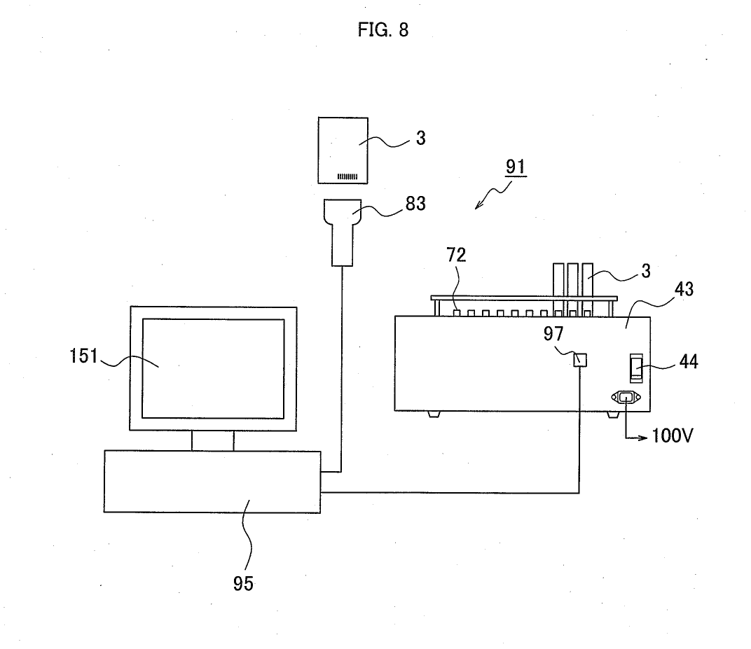

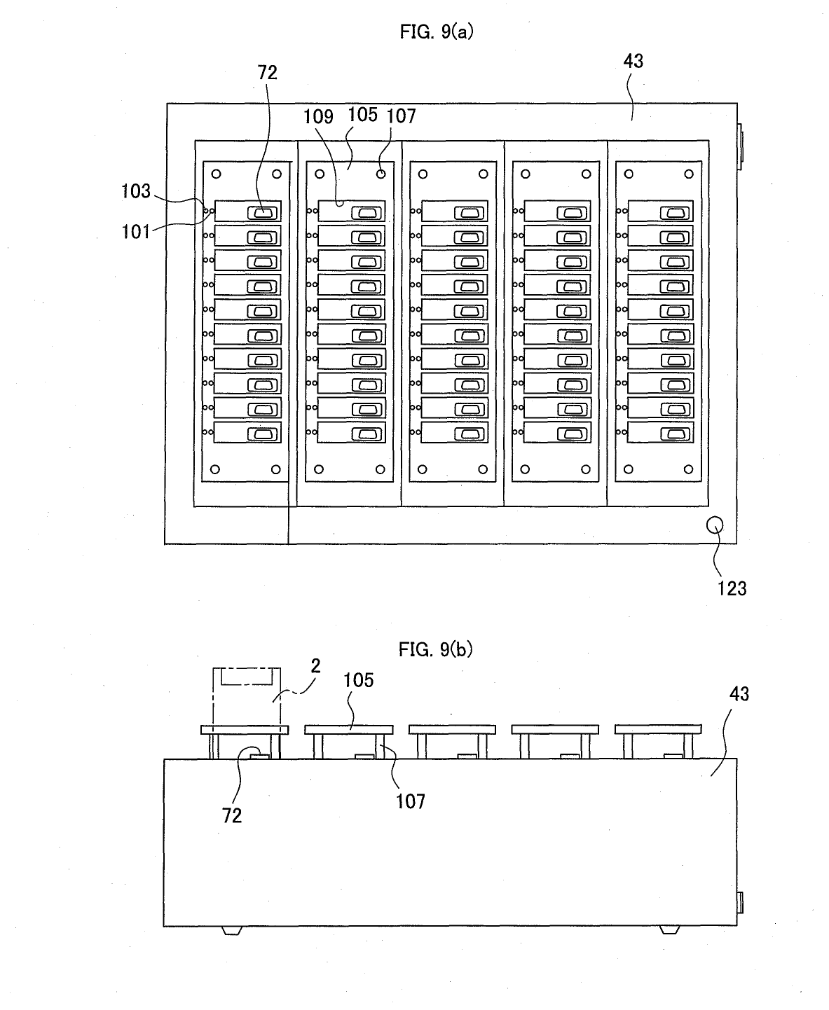

c) A configuration of a control system for collecting aspiration data from the aspiration state monitoring device 3 is now discussed with reference to FIGS. 8 and 9(a) and 9(b).

As illustrated in FIG. 8, a control system 91 for collecting aspiration data from the aspiration state monitoring device 3 according to this embodiment has a barcode reader 93 for reading the barcode provided on the aspiration state monitoring device 3, the base device 43 to which the plural aspiration state monitoring devices 3 are detachably attached, and a personal computer 95 for controlling the barcode reader 93 and the base device 43.

A power supply switch 44 is provided on the base device 43, and is connected with the personal computer 95 via a USB jack 97 which will be described later.

As illustrated in FIGS. 9(a) and 9(b), the base device 43 is a device to which up to 50 pieces of the aspiration state monitoring devices 3 can be attached, and 50 pieces of the base side connectors 72 are provided on the surface of the base device 43.

More specifically, the base side connectors 72 are arranged with 10 rows in the longitudinal direction and 5 rows in the transverse direction, and divided into 5 blocks having 10 rows in the longitudinal direction. Thus, each block of the base side connectors 72 can be separated from one another.

State indication LEDs 101 and charge indication LEDs 103 are provided on the surface of the base device 43 in correspondence with the respective base side connectors 72. The state indication LEDs 101 light up to guide positions to which the aspiration state monitoring devices 3 are attached, and flash at the time of abnormal condition. The charge indication LEDs 103 indicate charge condition. The charge indication LEDs 103 flash during charging, and light up when charging is completed.

Transparent guide plates 105 made of plastic material are attached to each block on the surface of the base device 43 with a predetermined clearance left from the surface.

The guide plates 105 are disposed in such a position that a hollow space can be formed (for example, at the height of one third of that of the aspiration state monitoring device 3) by using screws 107. A rectangular opening 109 having a shape corresponding to the external shape of the aspiration state monitoring device 3 is formed on each of the guide plates 105 at each position corresponding to the positions of the base side connectors 72.

d) An electrical configuration of the base device 43 is now explained with reference to FIG. 10.

As shown in FIG. 10, a base side electronic controller 111 for controlling the base device 43 is provided on the base device 43.

This base side electronic controller 111 has a known built-in A/D microcomputer 113, a USB controller 115, a charge circuit 117 for charging the aspiration state monitoring devices 3, and a communication circuit 119 for communicating with the aspiration state monitoring devices 3.

The USB controller 115 is connected with a USB jack 121, and further with the personal computer 95 via the USB jack 121.

The built-in A/D microcomputer 113 is connected with the respective state indication LEDs 101 and charge indication lamps 103. The charge circuit 117 and the communication circuit 119 are connected with the respective base side connectors 72.

More specifically, in each of the base side connectors 72 having the nine terminals 72a through 72i as discussed above, the three terminals 72a, 72b and 72c are connected with the charge circuit 117, and other terminals 72d and 72e are connected with the communication circuit 119.

Thus, when the device side lower connectors 45 of the aspiration state monitoring devices 3 are connected with the base side connectors 72, the rechargeable battery 69 is allowed to be charged via a pair of the terminals 72a and 72b of the terminals 72a through 72c connected with the charge circuit 117. Simultaneously, aspiration data can be received via a pair of the terminals 72d and 72e connected with the communication circuit 119.

The three terminals 72a through 72c connected with the charge circuit 117 are connected with the three terminals 45a through 45c of the device side lower connector 45, respectively. The two terminals 72a and 72b are connected with the plus side and minus side of the rechargeable battery 69, respectively, and the other terminal 72c is connected with a thermistor (not shown) provided in the vicinity of the rechargeable battery 69. That is, one end of the thermistor is connected with the other terminal 72c of the device side lower connector 45, and the other end of the thermistor is connected with the minus side of the rechargeable battery 69. When it is detected that the temperature of the rechargeable battery 69 has excessively increased, control to stop charging is executed.

According to this embodiment, data transmission and reception between the aspiration state monitoring devices 3 and the base device 43 is achieved by clock synchronous serial communication. Thus, aspiration data reception is normally completed within about 20 seconds per one aspiration state monitoring device 3.

The base device 43 further includes a charge start switch 123 for starting charging by manual operation, a power supply circuit 125 for supplying electric power to the base side electronic controller 111, and other components.

e) A method of using the aspiration state monitoring system, a method of using the control system, and the like are now discussed one by one.

(1) Aspiration State Monitoring Device Attachment Method

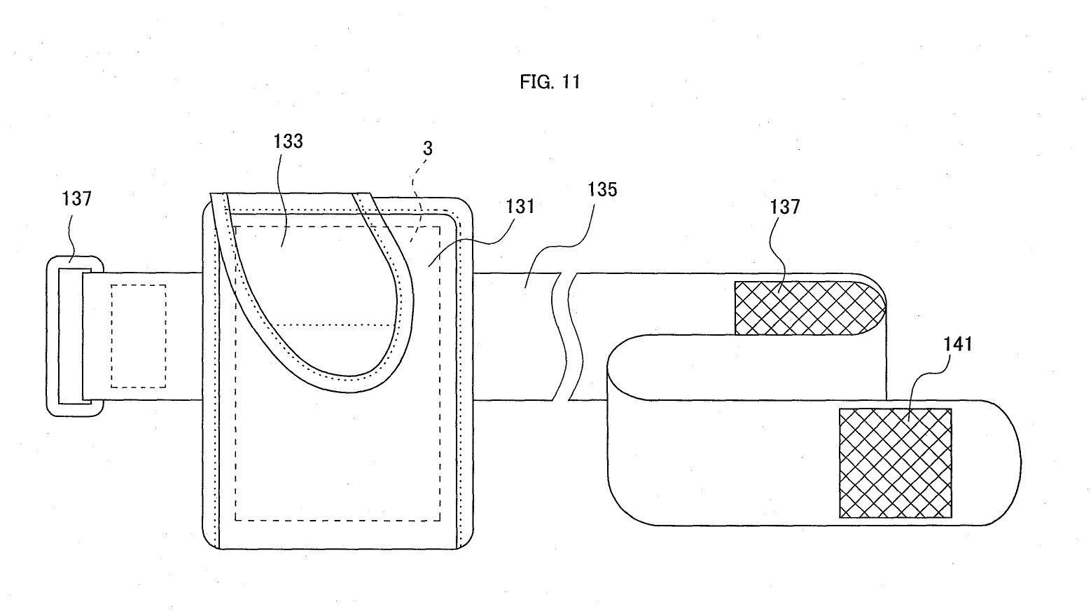

As illustrated in FIG. 11, the aspiration state monitoring device 3 according to this embodiment is accommodated in a substantially rectangular parallelepiped case 131 during use, for example.

An openable and closable cover 133 is provided on the upper portion of the case 131. A belt 135 for attaching the case 131 to the arm or the like is attached to one side (back side of the figure sheet) of the case 131. A ring 137 is provided at one end of the belt 135. A face fastener (male side) 139 is attached to the belt 135 around the middle on the other side. A face fastener (female side) 141 is attached to the end of the belt 135 on the other side.

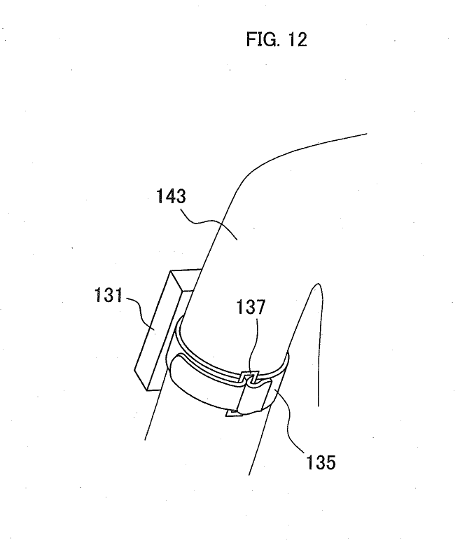

Thus, for attaching the case 131 accommodating the aspiration state monitoring device 3 to an arm, the belt 135 is wound around an arm 143 as illustrated in FIG. 12. Then, one end of the belt 135 is inserted through the ring 137 and folded, and the face fasteners 139 and 141 are brought into contact with each other to be fixed thereto.

(2) Aspiration State Measurement Method

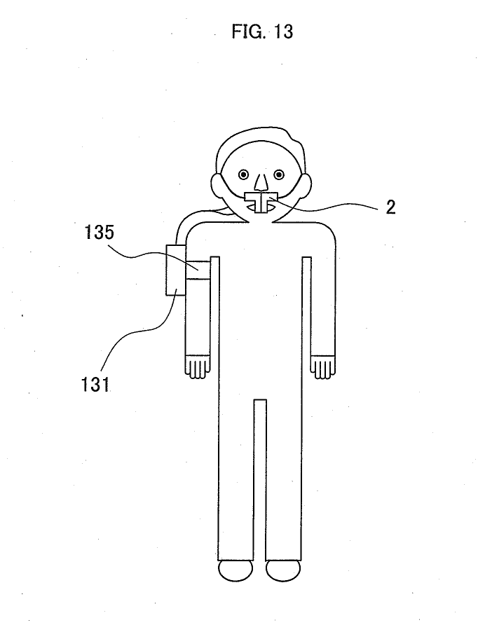

For measuring aspiration state of each examinee, the case 131 containing the aspiration state monitoring device 3 is wound around an arm of each examinee in the manner described above as illustrated in FIG. 13. In this step, the aspiration sensor 2 may be attached to the aspiration state monitoring device 3 either in advance or after the aspiration state monitoring device 3 is attached to an arm.

The sensor main body 5 of the aspiration sensor 2 is attached to area between a mouth and a nose by using a double face adhesive tape. More specifically, an adhesive surface of the connecting portion 7 is affixed to the area between the mouth and nose.

The affixation of the aspiration sensor 2, and the attachments of the aspiration state monitoring device 3 to an arm and of the aspiration sensor 2 to the aspiration state monitoring device 3 may be executed in any order appropriately determined.

When the sensor side connector 13 is attached to the device side upper connector 29, the circuit from the rechargeable battery 69 to the power supply circuit 71 is closed. This condition allows electric power to be supplied to the monitoring side electronic controller 27.

When the voltage of the rechargeable battery 69 is low in this step, the first LED 31 of the battery running out display device 65 lights up.

When the measurement start switch 41 is turned on, processing of the signal from the aspiration sensor 2 is initiated. More specifically, under the condition that the third LED 35 of the aspiration flow display device 67 is lighting, the second LED lights up when breath is inhaled, and the fourth LED 37 lights up when breath is exhaled.

After elapse of three minutes from the time when the measurement start switch 41 is turned on, the second through fourth LEDs 33 through 37 go out. Simultaneously, the time (time after elapse of three minutes) is read out from the real time clock 51 and stored in the flash memory 55. Also, the operation of storing the signal from the aspiration sensor 2 in the flash memory 55 is started.

Thus, accumulation of data indicating aspiration state starts after elapse of three minutes from the time when the measurement start switch 41 is turned on. It is therefore considered that the measurement substantially starts after the elapse of three minutes.

After elapse of 10 hours from the time when the measurement start switch 41 is turned on, the operation of storing signal indicating aspiration state ends to finish measurement of aspiration state.

By these operations, data necessary for checking aspiration state of an examinee is accumulated in the flash memory 55.

After completion of measurement, the aspiration sensor 2 is removed from a face, and the sensor side connector 13 is separated from the aspiration state monitoring device 3. By removing the sensor side connector 13, the circuit from the rechargeable battery 69 to the power supply circuit 71 is opened.

(3) Screening Method

As illustrated in FIG. 8, the respective aspiration state monitoring devices 3 separated from the aspiration sensors 2 are attached to the base device 43. Then, aspiration data is collected for screening.

More specifically, the control system 91 is constructed such that the base device 43 and the barcode reader 93 are connected with the personal computer 95, and respective processes are sequentially performed in accordance with instructions of control software included in the personal computer 95 in the following manner.

When the control software of the personal computer 95 is operated, a command for reading the barcode is issued. Thus, an operator reads the barcodes of the respective aspiration state monitoring devices 3 by using the barcode reader 93. That is, the process for inputting the IDs indicating the aspiration state monitoring devices 3 to the personal computer 95 is performed.

Then, the state indication LEDs 101 indicating attachment positions are turned on based on the inputted IDs to guide predetermined attachment positions of the base device 43 to which the aspiration state monitoring devices 3 are to be attached.

While looking at the lights of the state indication LEDs 101, the operator fits the device side lower connectors (female side) 45 provided on the aspiration state monitoring devices 3 into the base side connectors (male side) 72 from above such that the aspiration state monitoring devices 3 can be attached to the base device 43 as illustrated in FIG. 6.

By this attachment, the charge circuit 117 of the base device 43 is brought into connection with the rechargeable batteries 69 of the aspiration state monitoring devices 3. This condition allows the rechargeable batteries 69 to be charged. Apart from the circuit for charging, the communication circuit 119 of the base device 43 is brought into connection with the serial communication circuits 57 of the aspiration state monitoring devices 3. This condition allows communication between the aspiration state monitoring devices 3 and the base device 43.

In this step, the IDs of the aspiration state monitoring devices 3 are transmitted from the non-volatile memories 53 of the aspiration state monitoring devices 3 via the base device 43 to the personal computer 95.

Then, the personal computer 95 judges whether the IDs read from the barcode coincide with the IDs stored in the aspiration state monitoring devices 3 as illustrated in FIG. 8. When these IDs do not coincide, this fact is displayed on a display 151.

When the IDs coincide with one another, it is asked whether another attached aspiration state monitoring device 3 is present or not. When the aspiration state monitoring device 3 is present, the operations of reading the barcode and attaching the aspiration state monitoring device 3 to the instructed position are repeated in the similar manner.

After operations for all the aspiration state monitoring devices 3 are similarly performed, notification of operation completion is inputted to the personal computer 95.

In response to the inputted notification of completion, it is asked by the personal computer 95 whether charging and data reading are to be initiated or not. Thus, notification of initiation is inputted when process start is desired.

Based on the input of initiation notification of the charging and aspiration data reading, charging is started in response to a command from the personal computer 95. In addition, measurement date data and aspiration data are inputted from the aspiration state monitoring devices 3 to the personal computer 95.

In this step, a file corresponding to each aspiration state monitoring device 3 is automatically created by the personal computer 95. More specifically, a file having a file name constituted by the ID of the aspiration state monitoring device 3 read from the barcode and the measurement date is produced, and the aspiration data is stored in this file.

When the ID and the date are “1234” and “Dec. 25, 2005”, for example, a file having a file name of “0512251234” is automatically created.

As for the charging timing of the respective aspiration state monitoring devices 3, it is considered that the devices are charged in the order of attachment, for example. However, the devices may be charged at the same time. As for the transmission and reception timing of aspiration data, it is considered that aspiration data are sequentially transmitted and received in the order of attachment, for example. The charging and transmission and reception may be performed by the same aspiration state monitoring device 3 either simultaneously or in different timings.

The respective charging indication LEDs 103 flash during charging, and light up after completion of charging.

(4) Initialization Method

When the processes of charging and transmission and reception of aspiration data are normally completed, it is asked by the personal computer 95 whether initialization is executed or not. Thus, notification of initialization is inputted when initialization is desired.

Based on the initialization notification, a process of clearing the flash memory 55 of each aspiration state monitoring device 3 and a process of time adjustment of the real time clock 51 are performed in response to a command from the personal computer 95.

The initialization of the aspiration state monitoring device 3 is thus achieved by these processes. The initialized aspiration state monitoring device 3 is removed from the base device 43, and the aspiration sensor 2 is attached to the aspiration state monitoring device 3 such that measurement of aspiration state can be again conducted.

According to this embodiment, control to switch amplifiers to be used is performed apart from the above-described processes.

More specifically, after the aspiration sensor 2 is attached to the aspiration state monitoring device 3, signals from the aspiration sensor 2 are inputted to the charge AMP 59. The inputted signals are further inputted to the A/D built-in microcomputer 49 via the gain multiplication-by-1 AMP 61 and the gain multiplication-by-2 AMP 63.

Normally, signals from the gain multiplication-by-2 AMP 63 are processed and stored in the flash memory 55 as aspiration state signals. However, when the signals exceed 80% of the maximum acquisition of A/D conversion, the amplifiers are switched such that the signals from the gain multiplication-by-1 AMP 61 are processed.

By this structure, signals are prevented from reaching an upper limit of the maximum acquisition of A/D conversion, and therefore signal change corresponding to condition of aspiration can be detected at all times.

f) The advantages offered according to this embodiment are now discussed.

According to this embodiment, the plural aspiration state monitoring devices 3 are detachably attached to the base device 43. After attachment, the rechargeable batteries 69 of the aspiration state monitoring devices 3 can be charged by supplying electric power from the base device 43 through the connectors 45 and 72 of the base device 43 and the aspiration state monitoring devices 3. Also, aspiration data stored in the flash memories 55 of the aspiration state monitoring devices 3 can be collected and transmitted to the personal computer 95.

Thus, in case of screening for many examinees by using a number of the aspiration state monitoring devices 3, charging and aspiration data transmission and reception can be executed by attaching the aspiration state monitoring devices 3 after measurement to the base device 43. Accordingly, a significant advantage that screening can be performed by extremely easy operation with highly enhanced screening efficiency can be provided.

Particularly, each of the aspiration state monitoring devices 3 can be attached only by inserting the device lower connector 45 into the base side connector 72 and making connection therebetween according to this embodiment. This operation is extremely easy.

According to this embodiment, the base device 43 is divided into a plurality of blocks, and each of the blocks has the plural base side connectors 72. Thus, in case of failure in a circuit or the like within a certain block, only the corresponding block needs to be removed for repair or the like. Accordingly, maintenance is simplified.

According to this embodiment, the ID data of the barcode and the ID data stored in the flash memory 55 are read and compared. Thus, the advantage that operation error of mistaking another person's data for the correct data is prevented can be offered.

According to this embodiment, file name of a file automatically created for storing aspiration data contains ID (read from the barcode or read from the non-volatile memory 53) and date. Thus, an operation error of mistaking another person's data for a correct data can be also prevented in this aspect.

According to this embodiment, initialization and time adjustment (such as date setting) of the aspiration state monitoring device 3 are executed after the aspiration state monitoring device 3 is attached to the base device 43. Thus, an operation for re-using the aspiration state monitoring device 3 can be efficiently performed.

According to this embodiment, the circuit connecting the rechargeable battery 69 and the power supply circuit 71 is closed and the aspiration state monitoring device 3 comes to ON condition when the aspiration sensor 2 is attached to the aspiration state monitoring device 3. On the other hand, the circuit connecting the rechargeable battery 69 and the power supply circuit 71 is opened and the aspiration state monitoring device 3 comes to OFF condition when the aspiration sensor 2 is removed.

Thus, the aspiration state monitoring device 3 can be automatically turned on or off by attachment or detachment of the aspiration sensor 2. Accordingly, the structure of the device can be simplified and made compact. Moreover, operation can be securely performed with enhanced operability, and cost reduction can be achieved.

Particularly, according to this embodiment, charging is executed by attaching the aspiration state monitoring device to the base device 43, and the circuit between the rechargeable battery 69 and the power supply circuit 71 is opened after completion of measurement and removal of the aspiration sensor 2. In this case, there is less possibility that turning off of the power switch is forgotten than in case of a structure containing a separate power switch. Accordingly, the advantage that damage of the monitoring side electronic controller 27 is effectively prevented can be offered.

This embodiment therefore offers a remarkable advantage that operations required for screening of many examinees are considerably simplified by using the aspiration state monitoring system having the lightweight and simple structure described above.

According to this embodiment, operation conditions are notified through flashes of the second through fourth LEDs 33 through 37 according to use condition of the aspiration sensor 2 when the measurement start switch 41 is turned on under the condition that the aspiration sensor 2 is attached to the aspiration state monitoring device 3. Thus, whether the aspiration sensor 2 or the aspiration state monitoring device 3 is abnormal or not or other conditions can be recognized at the time of measurement start. Accordingly, an advantage that useless measurement such as measuring all night using a damaged device is avoided can be offered.

Additionally, since the second through fourth LEDs 33 through 37 go off after elapse of three minutes from the time when the measurement start switch 41 is turned on, the lights do not disturb sleep.

According to this embodiment, the amplifier to be used is switched according to a level of a sensor signal. Thus, sufficient accuracy of signal processing is secured, and a low-cost microcomputer having low processing capability can be used as a microcomputer used for control.

According to this embodiment, measurement ends after elapse of ten hours from start of measurement. Thus, only necessary data can be obtained.

According to this embodiment, the aspiration state monitoring device 3 is accommodated in the case 131, and the case 131 is attached to an arm by the belt 135. Thus, aspiration state can be appropriately measured even during sleep or when the examinee wakes up and moves during measurement.

Obviously, the present invention is not limited to the embodiment described herein, but may be practiced otherwise with various changes and modifications without departing from the technical scope of the invention.

(1) According to this embodiment, the personal computer separated from the base device is used as a device for controlling the base device. However, components such as controller and display may be incorporated in the base device such that aspiration data or the like can be collected by the base device.

(2) While aspiration is detected according to this embodiment, snoring can be detected as well as aspiration. In this case, such a component as a low-pass filter or a high-pass filter may be used.

More specifically, frequencies of output signals from the aspiration sensor are low at the normal aspiration action (no snoring), and frequencies of output signals from the aspiration sensor are high at the time of snoring. Thus, snoring can be detected by examining levels of frequencies of output signals from the aspiration sensor by using the low-pass filter or high-pass filter.

(3) In addition, a differential amplifier may be used instead of the charge AMP.

(4) Various detection elements such as thermocouple or other temperature detection elements other than the piezoelectric element may be used.

(5) The position to which the aspiration state monitoring device is attached is not limited to an arm but may be other positions such as an abdomen. Alternatively, the aspiration state monitoring device may be put around a pillow rather than attached.

1. An aspiration state monitoring device, comprising:
a rechargeable battery;
a memory that stores aspiration data indicating aspiration state of a living body; and
a monitoring side attachment unit detachably attached to a base device,
wherein the monitoring side attachment unit has a monitoring side charge conducting section that receives electric power from the base device and charges the rechargeable battery, and a monitoring side communication conducting section which transmits the aspiration data stored in the memory to the base device.

2. The aspiration state monitoring device according to claim 1, wherein the monitoring side attachment unit is a connector on the aspiration state monitoring device side connected with a connector on the base device side.

3. The aspiration state monitoring device according to claim 2, wherein the one connector of the aspiration state monitoring device has a terminal of the monitoring side charge conducting section and a terminal of the monitoring side communication conducting section.

4. The aspiration state monitoring device according to claim 1, wherein a barcode indicating an ID of the aspiration state monitoring device is provided on a surface of the aspiration state monitoring device.

5. The aspiration state monitoring device according to claim 1, wherein the memory included in the aspiration state monitoring device stores ID data indicating an ID of the aspiration state monitoring device.

6. A base device, comprising:
a base side attachment unit to which a plurality of aspiration state monitoring devices, each of which includes a rechargeable battery and a memory storing aspiration data indicating aspiration state of a living body, are detachably attached,
wherein the base side attachment unit has a base side charge conducting section that charges the rechargeable battery of each of the aspiration state monitoring devices, and a base side communication conducting section which collects the aspiration data stored in the memory of each of the aspiration state monitoring devices.

7. The base device according to claim 6, wherein the base side attachment unit is a connector on the base device side connected with a connector on the aspiration state monitoring device side.

8. The base device according to claim 7, wherein one connector of the base device has a terminal of the base side charge conducting section and a terminal of the base side communication conducting section.

9. The base device according to claim 6, wherein:
the base device is divided into a plurality of blocks; and
each of the blocks has a plurality of base side attachment units to which the respective aspiration state monitoring devices are detachably attached.

10. The base device according to claim 6, comprising a controller which controls the charging and the data collection.

11. The base device according to claim 10, wherein:
when ID data in a barcode provided on the aspiration state monitoring device to indicate an ID of the aspiration state monitoring device is read by the controller and when ID data stored in the memory of the aspiration state monitoring device to indicate the ID of the aspiration device monitoring device is read by the controller, the ID data in the barcode and the ID data in the memory are compared with each other.

12. The base device according to claim 10, wherein:
when ID data in a barcode provided on the aspiration state monitoring device to indicate an ID of the aspiration state monitoring device is read by the controller, a file name of a file for the aspiration data is automatically created based on the ID data and measurement date of the aspiration data.

13. The base device according to claim 10, wherein:
when ID data stored in the memory of the aspiration state monitoring device to indicate an ID of the aspiration state monitoring device is read by the controller, a file name of a file for the aspiration data is automatically created based on the ID data and measurement date of the aspiration data.

14. A control system comprising the aspiration state monitoring device according to claim 1, and the base device according to claim 6.

15. A method of using the control system according to claim 14, comprising:
attaching the aspiration state monitoring device to the base device;
charging the rechargeable battery of the aspiration state monitoring device by the base device via the base side charge conducting section and the monitoring side charge conducting section; and
collecting the aspiration data from the memory of the aspiration state monitoring device by the base device via the monitoring side communication conducting section and the base side communication conducting section.

16. The method of using the control system according to claim 14, comprising:
attaching the aspiration state monitoring device to the base device;
executing at least either initialization or time adjustment of the aspiration state monitoring device by the base device via the base side charge conducting section and the monitoring side charge conducting section.

17. An aspiration state monitoring device to which an aspiration sensor that outputs a signal corresponding to aspiration state of a living body is detachably attached to measure the aspiration state based on the signal from the aspiration sensor,
wherein electric continuity between a battery of the aspiration state monitoring device and an internal circuit of the aspiration state monitoring device is connected or disconnected by attachment or detachment of a connector of the aspiration sensor.

18. The aspiration state monitoring device according to claim 17, comprising a notifying unit that gives a notification corresponding to a use condition of the aspiration sensor when the aspiration sensor is attached to the aspiration state monitoring device.

19. The aspiration state monitoring device according to claim 18, wherein contents of the notification given by the notifying unit vary according to breath inhale and exhale actions of the living body.

20. The aspiration state monitoring device according to claim 19, wherein the notification given by the notifying unit stops after elapse of a predetermined initial time from measurement start of the aspiration state monitoring device.

21. The aspiration state monitoring device according to claim 20, wherein storing the measurement result in a memory starts after elapse of the predetermined initial time.

22. The aspiration state monitoring device according to claim 17, wherein magnification of an amplifier amplifying the signal from the aspiration sensor is automatically adjusted according to a condition of the signal.

23. The aspiration state monitoring device according to claim 17, wherein start time of measurement is recorded in case that the measurement is measurement of the aspiration state of the living body under a condition where the aspiration sensor is attached to the aspiration state monitoring device.

24. The aspiration state monitoring device according to claim 17, wherein measurement ends after elapse of a predetermined measurement time in case that the measurement is measurement of the aspiration state of the living body under a condition where the aspiration sensor is attached to the aspiration state monitoring device.

25. The aspiration state monitoring device according to claim 17, further comprising a case that accommodates the aspiration state monitoring device, and an attachment member that attaches the case to the living body.

26. The aspiration state monitoring device according to claim 1, which has the characteristics of the aspiration state monitoring device according to claim 17.

27. An aspiration state monitoring system, wherein the aspiration sensor is attached to the aspiration state monitoring device according to claim 17.
